(19) United States
(12) Patent Application Publication
(10) Pub. No.: US 2022/0221471 A1
(43) Pub. Date: Jul. 14, 2022

(54) Use of Reagent for Detecting Expression Level of Transferrin in Preparation of Diagnostic Reagent or Kit for Disease Caused by Imbalance of Intestinal Immune Tolerance

(71) Applicant: KUNMING INSTITUTE OF ZOOLOGY, CHINESE ACADEMY OF SCIENCES, YUNNAN (CN)

(72) Inventors: REN LAI, YUNNAN (CN); XIAOPENG TANG, YUNNAN (CN); MINGQIAN FANG, YUNNAN (CN)

(21) Appl. No.: 17/268,006

(22) PCT Filed: May 7, 2020
(86) PCT No.: PCT/CN2020/088867

§ 371 Date: Feb. 11, 2021

(30) Foreign Application Priority Data
Feb. 24, 2020 (CN) 202010111230.0

(51) Int. Cl. G01N 33/68; G01N 33/543

(52) U.S. Cl. CPC G01N 33/6893; G01N 2800/065; G01N 2333/79; G01N 33/54306

(57) ABSTRACT

The present disclosure provides use of a reagent for detecting an expression level of transferrin in the preparation of a diagnostic reagent or kit for a disease caused by imbalance of intestinal immune tolerance, and belongs to the technical field of medical molecular biology. In the present disclosure, the severity of intestinal inflammatory diseases is diagnosed according to the transferrin level, achieving the objective of the early diagnosis of the disease caused by imbalance of intestinal immune tolerance. In the present disclosure, the transferrin is used as a marker for imbalance of intestinal immune tolerance, featuring high specificity and sensitivity, and simple detection procedure.

The present application claims priority to Chinese Patent Application No. 202010111230.0 filed with China National Intellectual Property Administration (CNIPA) on Feb. 24, 2020 and entitled “USE OF REAGENT FOR DETECTING EXPRESSION LEVEL OF TRANSFERRIN IN PREPARATION OF DIAGNOSTIC REAGENT OR KIT FOR DISEASE CAUSED BY IMBALANCE OF INTESTINAL IMMUNE TOLERANCE”, the entire contents of which are incorporated herein by reference.

### TECHNICAL FIELD

The present disclosure relates to the technical field of medical molecular biology, and in particular to use of a reagent for detecting an expression level of transferrin in the preparation of a diagnostic reagent or kit for a disease caused by imbalance of intestinal immune tolerance.

### BACKGROUND

The intestinal tract is the first line of defense exposing to external food antigens and resisting against intestinal pathogens and infections from external antigens. A plurality of intestinal floras reside in the intestine. As an important immune system of the human body, the intestine not only efficiently recognizes harmful (external pathogenic bacteria) and harmless antigens (intestinal symbiotic bacteria and food antigens), but also effectively challenges the stimulation of a plurality of antigenic substances (lipopolysaccharides, lipoteichoic acid, bacterial DNA, and the like) from the intestinal microbial metabolism and efficiently maintains the balance of intestinal immune tolerance. Imbalance of intestinal immune tolerance may lead to the onset of severe inflammatory bowel disease (IBD).

Inflammatory suppressor cells such as immunotolerant dendritic cells (CD103⁺CD11b⁺DC), regulatory T cells (Treg) and regulatory B cells (Treg) play a crucial role in maintaining the balance of intestinal immune tolerance, but molecular mechanisms thereof are still unclear. Currently, there is no clinically accurate and effective marker for detecting diseases related to intestinal immune imbalance.

### SUMMARY

An objective of the present disclosure is to provide use of a reagent for detecting an expression level of transferrin in the preparation of a diagnostic reagent or kit for a disease caused by imbalance of intestinal immune tolerance. The present disclosure may be used to accurately and effectively detect diseases related to intestinal immune imbalance.

To achieve the above objective, the present disclosure provides the following technical solutions:

The present disclosure provides use of a reagent for detecting an expression level of transferrin in the preparation of a diagnostic reagent or kit for a disease caused by imbalance of intestinal immune tolerance.

The present disclosure provides use of a reagent for detecting an expression level of transferrin in the diagnosis of a disease caused by imbalance of intestinal immune tolerance.

Preferably, the disease caused by imbalance of intestinal immune tolerance may include ulcerative colitis.

The present disclosure provides use of transferrin as a marker in the diagnosis of a disease caused by imbalance of intestinal immune tolerance.

The present disclosure provides an ELISA diagnostic kit for a disease caused by imbalance of intestinal immune tolerance using transferrin as a marker.

Preferably, the kit may include a rabbit anti-transferrin polyclonal antibody.

Preferably, the kit may further include: a well plate, a coating buffer, a wash buffer, a blocking buffer, an anti-rabbit IgG secondary antibody, a chromogenic substrate, and a stop buffer.

Preferably, the coating buffer may include phosphate buffered saline (PBS); the wash buffer may include phosphate buffered saline with Tween (PBST); the blocking buffer may include bovine serum albumin solution, and the bovine serum albumin solution may use the PBS as a solvent; the chromogenic substrate may include a 3,3′,5,5′-tetramethylbenzidine (TMB) solution; and the stop buffer may include an aqueous sulfuric acid solution.

The present disclosure has the following beneficial effects: The present disclosure provides use of a reagent for detecting an expression level of transferrin in the preparation of a diagnostic reagent or kit for a disease caused by imbalance of intestinal immune tolerance. In the present disclosure, the severity of intestinal inflammatory-diseases is diagnosed according to the transferrin level, achieving the objective of the early diagnosis of the disease caused by imbalance of intestinal immune tolerance. In the present disclosure, the transferrin is used as a marker for imbalance of intestinal immune tolerance, featuring high specificity and sensitivity, and simple detection procedure.

### BRIEF DESCRIPTION OF THE DRAWINGS

FIG. 1 illustrates the content of transferrin (Tf) in anticoagulant blood of patients with ulcerative colitis and normal subjects in Example 2;

FIG. 2 illustrates the Western blot result of levels of the transferrin in the colonic tissues of the patients with ulcerative colitis in Example 2;

FIG. 3 illustrates the statistical results of levels of the transferrin in the colonic tissues of the patients with ulcerative colitis in Example 2;

FIG. 4 illustrates the Western blot results of transferrin and important indicators for intestinal immune tolerance (retinal dehydrogenase ALDH1A2, CCL22, TGF-β1 and IL-10) in intestinal tissues and all segments (duodenum (D), jejunum (J), ileum (I), cecum (C1) and colon (C2)) of mesenteric lymph nodes of ordinary SPF mice and germ-free (GF) mice in Example 3;

FIG. 5 illustrates the Western blot statistical results of the Tf of the intestinal tissues and all segments of mesenteric lymph nodes of the ordinary SPF mice and germ-free (GF) mice in Example 3;

FIG. 6 illustrates the Western blot statistical results of the ALDH1A2 of the intestinal tissues and all segments of mesenteric lymph nodes of the ordinary SPF mice and germ-free (GF) mice in Example 3;

FIG. 7 illustrates the Western blot statistical results of the CCL22 of the intestinal tissues and all segments of mesenteric lymph nodes of the ordinary SPF mice and germ-free (GF) mice in Example 3;

FIG. 8 illustrates the Western blot statistical results of the TGF-β1 of the intestinal tissues and all segments of mesenteric lymph nodes of the ordinary SPF mice and germ-free (GF) mice in Example 3;

FIG 9 illustrates the Western blot statistical results of the IL-10 of the intestinal tissues and all segments of mesenteric lymph nodes of the ordinary SPF mice and germ-free (GF) mice in Example 3;

FIG. 10 illustrates the Western blot results of transferrin and important indicators for intestinal immune tolerance (retinal dehydrogenase ALDH1A2, CCL22, TGF-β1 and IL-10) in intestinal tissues and all segments (duodenum (D), jejunum (J). ileum (I), cecum (C1) and colon (C2)) of mesenteric lymph nodes of ordinary SPF mice (NC) and SPF mice fed with compound antibiotics (Abs) in Example 4;

FIG. 11 illustrates the Western blot statistical results of the Tf of the intestinal tissues and all segments of mesenteric lymph nodes of the SPF mice (NC) and SPF mice fed with compound antibiotics in Example 4;

FIG. 12 illustrates the Western blot statistical results of the ALDH1A2 of the intestinal tissues and all segments of mesenteric lymph nodes of the SPF mice (NC) and SPF mice fed with compound antibiotics in Example 4;

FIG. 13 illustrates the Western blot statistical results of the CCL22 of the intestinal tissues and all segments of mesenteric lymph nodes of the SPF nice (NC) and SPF mice fed with compound antibiotics in Example 4;

FIG. 14 illustrates the Western blot statistical results of the TGF-β1 of the intestinal tissues and all segments of mesenteric lymph nodes of the SPF mice (NC) and SPF mice fed with compound antibiotics in Example 4;

FIG. 15 illustrates the Western blot statistical results of the IL-10 of the intestinal tissues and all segments of mesenteric lymph nodes of the SPF mice (NC) and SPF mice fed with compound antibiotics in Example 4;

FIG. 16 illustrates the effects of transferrin knockdown (SH) and feeding compound antibiotics (Abs) on dendritic cells (CD103⁺CD11b⁺, CD103⁺CD11b⁻, CD103⁻CD11b⁻, and CD103⁻CD11b⁺) in all segments (duodenum (D), jejunum (J), ileum (I), cecum (C1), and colon (C2)) of mouse intestinal tissue (Gut);

FIG. 17 illustrates the effects of transferrin knockdown (SH) and feeding compound antibiotics (Abs) on dendritic cells (CD103⁺CD11b⁺, CD103⁺CD11b⁻, CD103⁻CD11b⁻, and CD103⁻CD11b⁺) in all segments (duodenum (D), jejunum (J), ileum (I), cecurn (C1), and colon (C2)) of mouse mesenteric lymph nodes (gLN);

FIG. 18 illustrates the statistical results of transferrin knockdown (SH) and feeding compound antibiotics (Abs) against important indicator for immune tolerance, differentiation of dendritic cells (CD103⁺CD11b⁺, CD103⁺CD11b⁻, CD103⁻CD11b⁻, and CD103⁻CD11b⁺) in all segments of mouse intestinal tissue (Gut);

FIG. 19 illustrates the statistical results of transferrin knockdown (SH) and feeding compound antibiotics (Abs) against important indicator for immune tolerance, differentiation of dendritic cells (CD103⁺CD11b⁺, CD103⁺CD11b⁻, CD103⁻CD11b⁻, and CD103⁻CD11b⁺) in all segments of mouse mesenteric lymph nodes (gLN);

FIG. 20 illustrates the effects of transferrin knockdown (SH) and feeding compound antibiotics (Abs) on FOXP3⁺RORγT⁺ Treg and FOXP3⁺ Treg in all segments (duodenum (D), jejunum (J). ileum (I), cecum (C1), and colon (C2)) of mouse intestinal tissue (Gut);

FIG. 21 illustrates the effects of transferrin knockdown (SH) and feeding compound antibiotics (Abs) on FOXP3⁺RORγT⁺ Treg and FOXP3⁺ Treg in all segments (duodenum (D), jejunum (J), ileum (I), cecum (C1), and colon (C2)) of mouse mesenteric lymph nodes (gLN);

FIG. 22 illustrates the statistical results of effects of transferrin knockdown (SH) and feeding compound antibiotics (Abs) on FOXP3⁺RORγT⁺ Treg in all segments of mouse intestinal tissue (Gut);

FIG. 23 illustrates the statistical results of effects of transferrin knockdown (SH) and feeding compound antibiotics (Abs) on FOXP3⁺Treg in all segments of mouse intestinal tissue (Gut);

FIG. 24 illustrates the statistical results of effects of transferrin knockdown (SH) and feeding compound antibiotics (Abs) on FOXP3⁺RORγT⁺ Treg in all segments of mesenteric lymph nodes (gLN);

FIG. 25 illustrates the statistical results of effects of transferrin knockdown (SH) and feeding compound antibiotics (Abs) on FOXP3⁺ Treg in all segments of mouse mesenteric lymph nodes (gLN).

### DETAILED DESCRIPTION

The present disclosure provides use of a reagent for detecting an expression level of transferrin in the preparation of a diagnostic reagent or kit for a disease caused by imbalance of intestinal immune tolerance; the disease caused by imbalance of intestinal immune tolerance may preferably include ulcerative colitis; the transferrin may preferably be transferrin in plasma and/or intestinal tissue.

The present disclosure diagnoses diseases related to imbalance of intestinal immune tolerance based on the down-regulation of levels of transferrin in plasma and colonic tissues of patients with ulcerative colitis, a disease caused by imbalance of intestinal immune tolerance. The down-regulation of transferrin level is related to the destruction of homeostasis of intestinal immune tolerance. The present disclosure detects the transferrin level in patient's plasma and intestinal tract as a marker for early diagnosis and monitoring of diseases related to intestinal immune tolerance imbalance.

The present disclosure provides an ELISA diagnostic kit for a disease caused by imbalance of intestinal immune tolerance using transferrin as a marker; the kit may preferably include a rabbit anti-transferrin polyclonal antibody. The present disclosure has no special restrictions on preparation methods of the rabbit anti-transferrin polyclonal antibody, as long as conventional methods in the art may be used.

In the present disclosure, the kit may preferably further include: a well plate, a coating buffer, a wash buffer, a blocking buffer, an anti-rabbit IgG secondary antibody, a chromogenic substrate, and a stop buffer.

In the present disclosure, the coating buffer may include PBS, the PBS may preferably have a pH value of 9.6, solutes of the PBS may include Na₂CO₃ and NaHCO₃, and the PBS may preferably have a solute concentration of 0.05 M; the wash buffer may include PBST, and the Tween in the PBST may preferably have a volume percentage of 0.5%; the blocking buffer may include bovine serum albumin solution, the bovine serum albumin solution may use the PBS as a solvent, and the bovine serum albumin in the bovine serum albumin solution may have a mass percentage of 1%; the chromogenic substrate may include a 3,3′,5,5′-tetramethylbenzidine (TMB) solution; the stop buffer may include an aqueous sulfuric acid solution, and the aqueous sulfuric acid solution may have a sulfuric acid concentration of 2 M.

A method for using the kit of the present disclosure may preferably include the following steps:

Plasma samples or intestinal tissues collected from patients with disease caused by imbalance of intestinal immune tolerance are diluted with coating buffer and plated on a 96-well plate; after blocking treatment, the transferrin in wells of the plate is specifically adsorbed with a rabbit anti-transferrin polyclonal antibody, and developed with a horseradish peroxidase (HRP)-conjugated secondary antibody. The level of transferrin in the sample is determined by using a standard curve. The severity of intestinal inflammatory diseases is diagnosed according to the transferrin level, achieving the objective of the early diagnosis of the disease caused by imbalance of intestinal immune tolerance.

The technical solutions of the present disclosure will be described below clearly and completely with reference to the examples of the present disclosure, It is clear that the described examples are only a part of, not all of, the examples of the present disclosure. All other examples obtained by those of ordinary skill in the art based on the examples of the present disclosure without creative efforts shall fall within the scope of the present disclosure.

### EXAMPLE 1

Transferrin assay kit.

The determination method was conventional enzyme-linked immunosorbent assay (ELISA), and the detection steps were as follows:

1) 1 μl each of 1000-fold diluted plasma of a patient with ulcerative colitis and a normal subjected was mixed with 99 μl of fixing solution (50 mM carbonate buffer, pH 9.6), added to a 96-well plate (Nunc, Denmark), and coated overnight at 4° C.

2) The next day, the solution was discarded from the wells, and the plasma was washed thrice with 0.1 M PBS and blocked; homemade a rabbit anti-transferrin polyclonal antibody (10 μg/ml) was added thereto and incubated for 60 min at 37° C. The preparation process of the rabbit anti-transferrin polyclonal antibody was as follows: 500 μg of human transferrin (Sigma) was dissolved in 1 mL of Freund's Complete Adjuvant (Sigma) and subcutaneously injected at a plurality of sites of a rabbit (male, 2 kg). At 14, 28 and 42 days after the first immunization, 250 μg of human transferrin dissolved in Freund's Incomplete Adjuvant (Sigma) was subcutaneously injected into the rabbit in the same manner. Finally, after the blood was collected from the ear vein, the rabbit anti-transferrin polyclonal antibody was isolated and purified with an antibody isolation kit (Amersham Biosciences, Piscataway, NI, USA).

3) After washing unconjugated antibody with wash buffer, a HRP-conjugated anti-rabbit IgG secondary antibody (KPL, USA) was added, and finally TMB was used for color reaction.

4) A series of serial dilutions of standard transferrin were used as a standard curve to obtain the concentration of transferrin in the measured plasma sample. The specific operation process was to prepare the purchased pure human transferrin (Sigma) into 7 standard concentration gradients, i.e., 20, 10, 5, 2.5, 1.25, 0.625, and 0.3125 mg/ml.

### EXAMPLE 2

Detection of levels of transferrin in plasma and colonic tissues of patients with ulcerative colitis and normal subjects by ELISA.

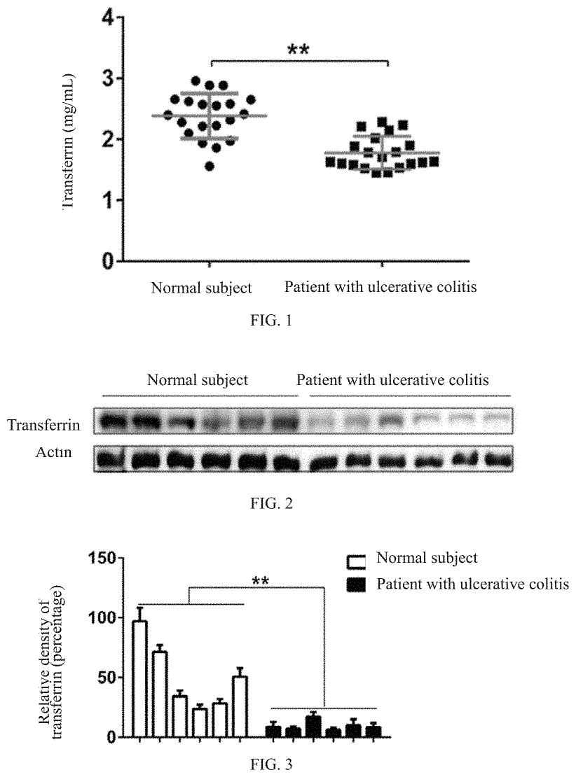

The plasma was collected from 20 patients with ulcerative colitis and normal subjects in a hospital, and the plasma and 3.8% (mass: volume) sodium citrate were mixed in a ratio of 1:9 (volume: volume) to obtain anticoagulant blood. Levels of transferrin in anticoagulant blood were detected according to the method in Example 1. The detection results are shown in FIG. 1. The plasma transferrin levels of patients with ulcerative colitis show a significant decrease (p<0.01). Tf stands for transferrin.

Ground colonic tissues of normal subjects and patients with ulcerative colitis (n=20) were subjected to Western blot using the anti-transferrin antibody described in Example 1. The results are shown in FIGS. 2 and 3. FIG. 2 illustrates the Western blot result of levels of the transferrin in the colonic tissues of the patients with ulcerative colitis; FIG. 3 illustrates the Western blot statistical results of levels of the transferrin in the colonic tissues of the patients with ulcerative colitis, and the statistical results come from 5 independent repeated experiments of 20 patients. ** p<0.01; the levels of transferrin in colonic tissues of the patients with ulcerative colitis decrease significantly (p<0.01).

### EXAMPLE 3

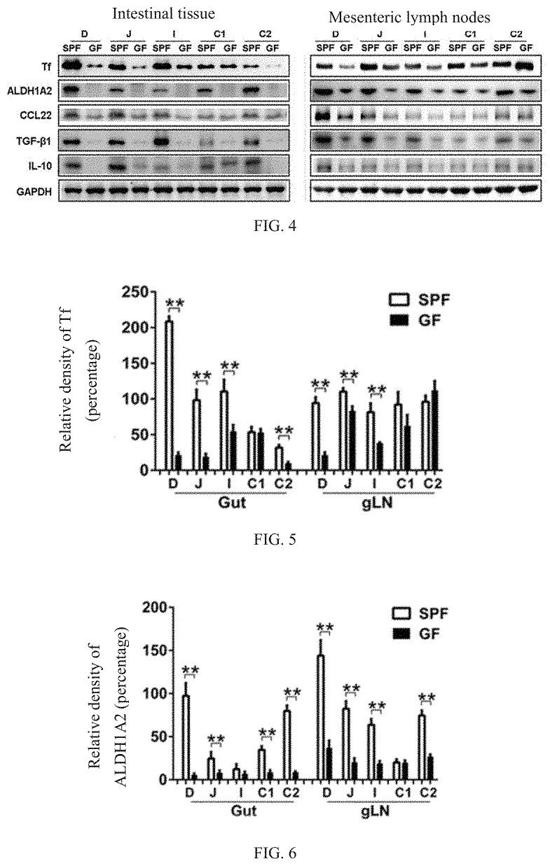

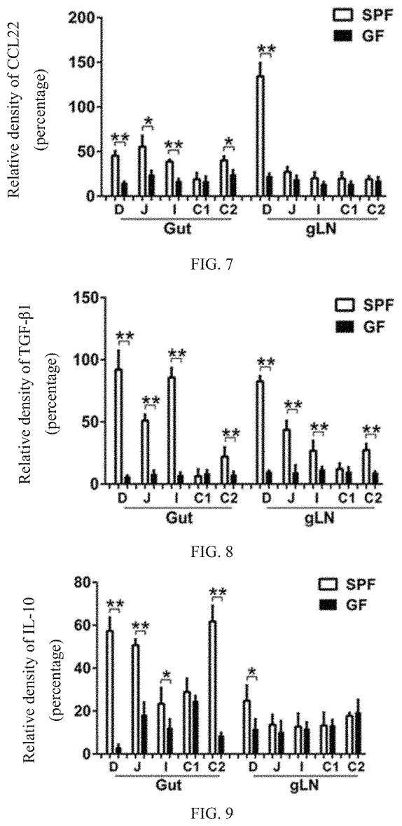

To further verify the role of transferrin in maintaining intestinal immune tolerance, the present disclosure determined the content of transferrin and important indicators for intestinal immune tolerance (retinal dehydrogenase ALDH1A2, CCL22, TGF-β1 and IL-10) in intestinal tissues and all segments {duodenum (D), jejunum (J), ileum (I), cecum (C1) and colon (C2){ of mesenteric lymph nodes of ordinary SPF mice and germ-free (GF) mice. The results are as follows: compared with the SPF mice, the content of transferrin and important indicators for intestinal immune tolerance (ALDH1A2, CCL22, TGF-β1 and IL-10) decreases in the intestinal tissues and mesenteric lymph nodes of the germ-free mice (FIGS. 4 to 9, *p<0.05; **p<0.01), where: FIG. 4 illustrates the Western blot results of the transferrin and important indicators for intestinal immune tolerance (retinal dehydrogenase ALDH1A2, CCL22, TGF-β1 and IL-10) in intestinal tissues and all segments (duodenum (D), jejunum (J), ileum (I), cecum (C1) and colon (C2)) of mesenteric lymph nodes of the ordinary SPF mice and the germ-free (GF) mice; FIG. 5 illustrates the Western blot statistical results of the Tf of the intestinal tissues and all segments of mesenteric lymph nodes of the ordinary SPF mice and the germ-free (OF) mice; FIG. 6 illustrates the Western blot statistical results of the ALDH1A2 of the intestinal tissues and all segments of mesenteric lymph nodes of the ordinary SPF mice and the germ-free (GF) mice; FIG. 7 illustrates the Western blot statistical results of the CCL22 of the intestinal tissues and all segments of mesenteric lymph nodes of the ordinary SPF mice and the germ-free (GF) mice; FIG. 8 illustrates the Western blot statistical results of the TGF-β1 of the intestinal tissues and all segments of mesenteric lymph nodes of the ordinary SPF mice and the germ-free (GF) mice; FIG. 9 illustrates the Western blot statistical results of the IL-10 of the intestinal tissues and all segments of mesenteric lymph nodes of the ordinary SPF mice and the germ-free (GF) mice.

### EXAMPLE 4

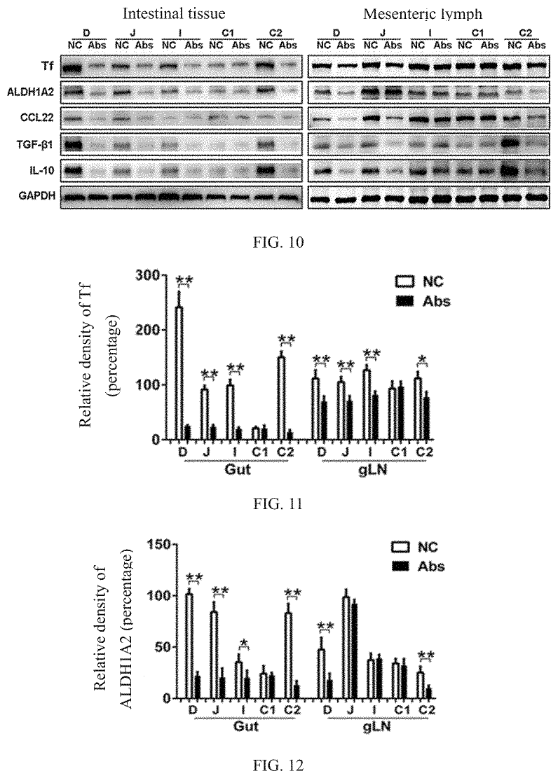

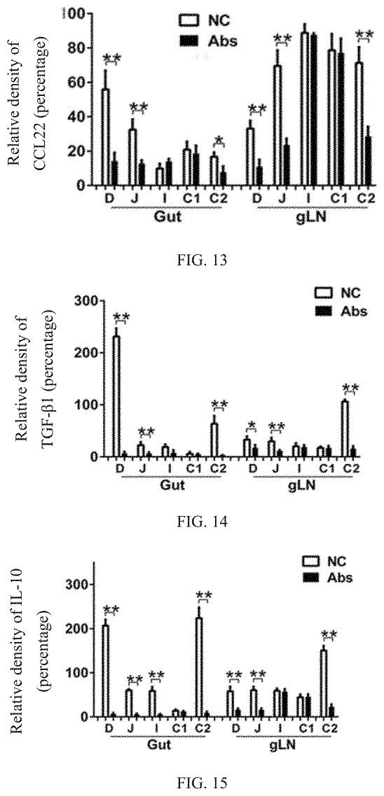

To further verify the role of transferrin in maintaining intestinal immune tolerance, the present disclosure determined the content of transferrin and important indicators for intestinal immune tolerance (retinal dehydrogenase ALDH1A2, CCL22, TOT-β1 and IL-10) in intestinal tissues and all segments of mesenteric lymph nodes (duodenum (D), jejunum (J), ileum (I), cecum (C1) and colon (C2)) of SPF mice (NC) and SPF mice led with compound antibiotics (Abs; the specific operation was to prepare an aqueous solution of ampicillin (1 g/L), streptomycin (1 g/L), metronidazole (0.5 g/ml) and vancomycin (1 g/L) to feed the mice for three weeks). The results are as follows: compared with the control mice (NC), the content of transferrin and important indicators for intestinal immune tolerance (ALDH1A2, CCL22, TGF-β1 and IL-10) decreases in the intestinal tissues and mesenteric lymph nodes of the mice fed with compound antibiotics (FIGS. 10 to 15, *p<0.05; **p<0.01), where: FIG. 10 illustrates the Western blot results of transferrin and important indicators for intestinal immune tolerance (retinal dehydrogenase ALDH1A2, CCL22, TGF-β1 and IL-10) in intestinal tissues and all segments of mesenteric lymph nodes (duodenum (D), jejunum (J), ileum (I), cecum (C1) and colon (C2)) of the ordinary SPF mice (NC) and the SPF mice fed with compound antibiotics; FIG. 11 illustrates the Western blot statistical results of the Tf of the intestinal tissues and all segments of mesenteric lymph nodes of the SPF mice (NC) and the SPF mice fed with compound antibiotics; FIG. 12 illustrates the Western blot statistical results of the ALDH1A2 of the intestinal tissues and all segments of mesenteric lymph nodes of the SPF mice (NC) and the SPF mice fed with compound antibiotics; FIG. 13 illustrates the Western blot statistical results of the CCL22 of the intestinal tissues and all segments of mesenteric lymph nodes of the SPF mice (NC) and the SPF mice fed with compound antibiotics; FIG. 14 illustrates the Western blot statistical results of the TGF-β1 of the intestinal tissues and all segments of mesenteric lymph nodes of the SPF mice (NC) and the SPF mice fed with compound antibiotics; FIG. 15 illustrates the Western blot statistical results of the IL-10 of the intestinal tissues and all segments of mesenteric lymph nodes of the SPF mice (NC) and the SPF mice fed with compound antibiotics.

### EXAMPLE 5

To further verify the effects of transferrin on indicators for intestinal immune tolerance, the present disclosure determined the effects of transferrin knockdown (SH) and compound antibiotics (Abs; the construction method was the same as that in Example 4) on important indicator for immune tolerance, differentiation of dendritic cells (CD103⁺CD11b⁺ DC) and regulatory T cells (FOXP3⁺RORγT⁺ Treg and FOXP3⁺Treg), the specific grouping and administration methods were as follows: control group (NC, normal saline group), transferrin knockdown group (SH), viral administration group (the amount of virus administered by tail vein injection was 10⁷ transducing units (TU)), and the compound antibiotics feeding group (Abs).

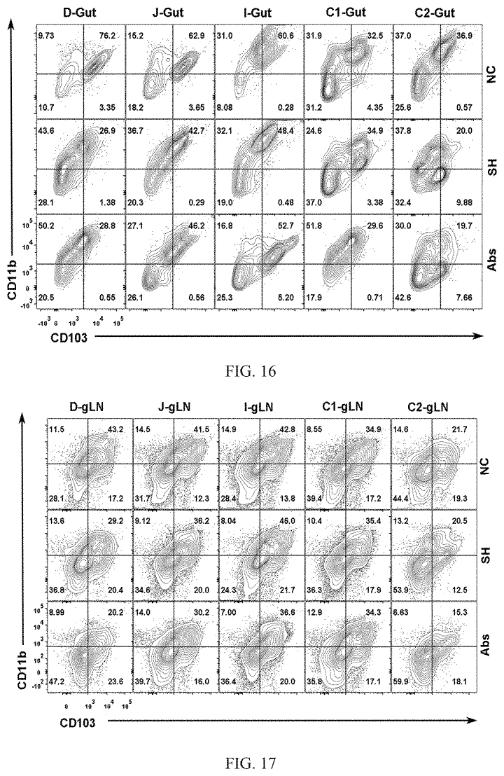

The results were as follows: Compared with the control group (normal saline group), the differentiation of dendritic cells (CD103⁺CD11b⁺) and regulatory T cells (FOXP3⁺RORγT⁺ Treg and FOXP3⁺ Treg) significantly decreased in the transferrin knockdown group (SH) and the compound antibiotics feeding group (FIGS. 16 to 25, p<0.01).

Herein, FIG. 16 illustrates the effects of transferrin knockdown (SH) on dendritic cells (CD103⁺CD11b⁺, CD103⁺CD11b⁻, CD103⁻CD11b⁻, and CD103⁻CD11b⁺) in mouse intestinal tissues (Gut) and all segments (duodenum (D), jejunum (J), ileum (I), cecum (C1), and colon (C2)) of mesenteric lymph nodes (gLN);

FIG. 17 illustrates the effects of feeding compound antibiotics (Abs) dendritic cells (CD103⁺CD11b⁺, CD103⁺CD11b⁻, CD103⁻CD11b⁻, and CD103⁻CD11b⁺) in mouse intestinal tissues (Gut) and all segments (duodenum (D), jejunum (J), ileum (I), cecum (C1), and colon (C2)) of mesenteric lymph nodes (gLN);

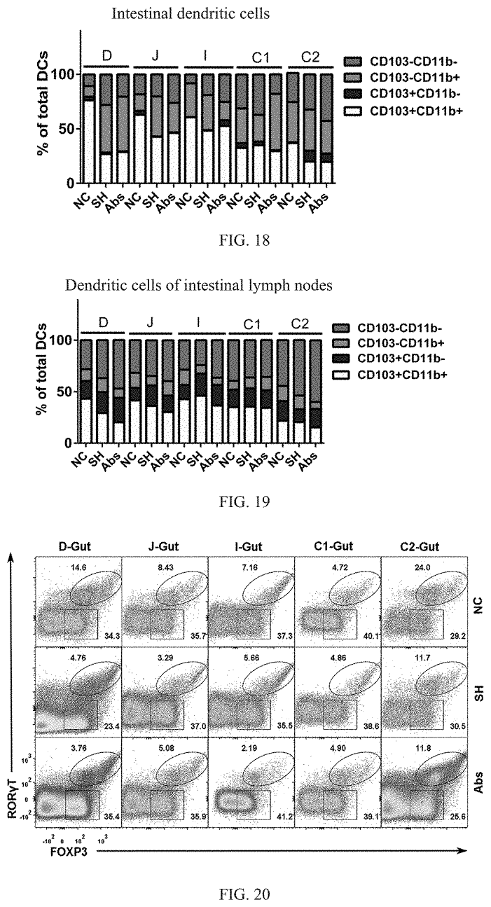

FIG. 18 illustrates the statistical results of transferrin knockdown (SH) against important indicator for immune tolerance, differentiation of dendritic cells (CD103⁺CD11b⁺ DC) and regulatory T cells (FOXP3⁺RORγT⁺Treg and FOXP3⁺ Treg) in mouse intestinal tissues (Gut) and all segments of mesenteric lymph nodes (gLN);

FIG. 19 illustrates the statistical results of feeding compound antibiotics (Abs) against important indicator for immune tolerance, differentiation of dendritic cells (CD103⁺CD11b⁺ DC) and regulatory T cells (FOXP3⁺RORγT⁺ Treg and FOXP3⁺ Treg) in mouse intestinal tissues (Gut) and all segments of mesenteric lymph nodes (gLN);

FIG. 20 illustrates the effects of transferrin knockdown (SH) on FOXP3⁺RORγT⁺ Treg and FOXP3⁺ Treg in mouse intestinal tissues (Gut) and all segments (duodenum (D), jejunum (J), ileum (I), cecum (C1), and colon (C2)) of mesenteric lymph nodes (gLN);

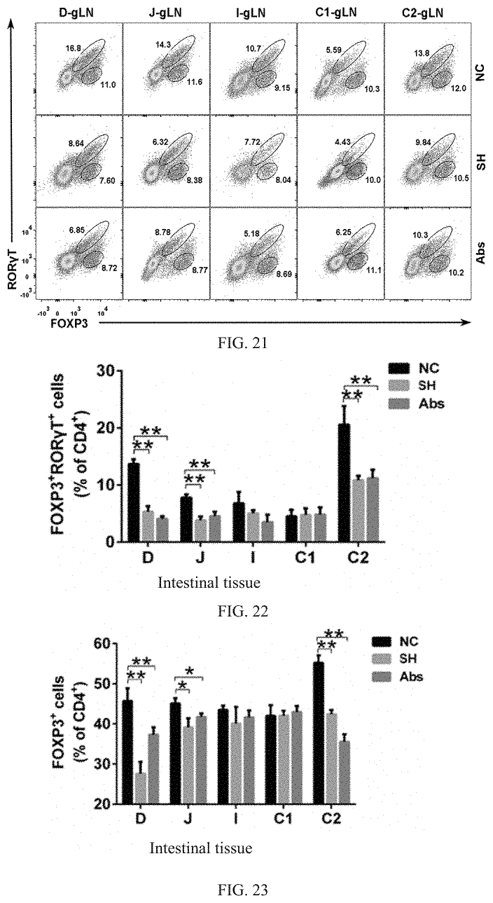

FIG. 21 illustrates the effects of feeding compound antibiotics (Abs) on FOXP3⁺RORγT⁺ Treg and FOXP3⁺ Treg in mouse intestinal tissues (Gut) and all segments (duodenum (D), jejunum (J), ileum (I), cecum (C1), and colon (C2)) of mesenteric lymph nodes (gLN);

FIG. 22 illustrates the statistical results of effects of transferrin knockdown (SH) on FOXP3⁺RORγT⁺ Treg in all segments of mouse intestinal tissue (Gut);

FIG. 23 illustrates the statistical results of effects of transferrin knockdown (SH) on FOXP3⁺Treg in all segments of mouse intestinal tissue (Gut);

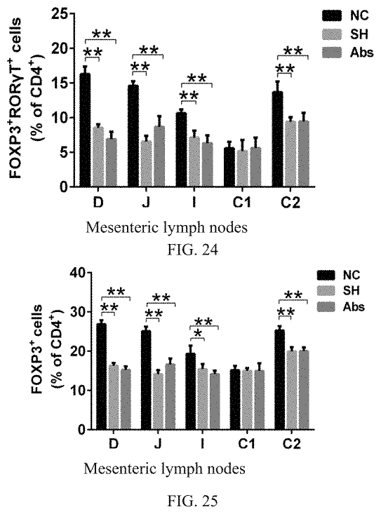

FIG. 24 illustrates the statistical results of effects of feeding compound antibiotics (Abs) on FOXP3⁺RORγT⁺ Treg in all segments of mouse intestinal tissue (Gut);

FIG. 25 illustrates the statistical results of effects of feeding compound antibiotics (Abs) on FOXP3⁺Treg in all segments of mouse intestinal tissue (Gut).

The above is only the preferred examples of the present disclosure; it should be noted that several improvements and modifications can also be made by those of ordinary skill in the art without departing from the principles of the present disclosure, and these improvements and modifications should also be regarded as the protection scope of the present disclosure.

What is claimed is:

1. Use of a reagent for detecting an expression level of transferrin in the preparation of a diagnostic reagent or kit for a disease caused by imbalance of intestinal immune tolerance.

2. Use of a reagent for detecting an expression level of transferrin in the diagnosis of a disease caused by imbalance of intestinal immune tolerance.

3. The use according to claim 1, wherein the disease caused by imbalance of intestinal immune tolerance comprises ulcerative colitis.

4. Use of transferrin as a marker in the diagnosis of a disease caused by imbalance of intestinal immune tolerance.

5. An ELISA diagnostic kit for a disease caused by imbalance of intestinal immune tolerance using transferrin as a marker.

6. The kit according to claim 5, wherein the kit comprises a rabbit anti-transferrin polyclonal antibody.

7. The kit according to claim 5, wherein the kit further comprises: a well plate, a coating buffer, a wash buffer, a blocking buffer, an anti-rabbit IgG secondary antibody, a chromogenic substrate, and a stop buffer.

8. The kit according to claim 7, wherein the coating buffer comprises phosphate buffered saline (PBS); the wash buffer comprises phosphate buffered saline with Tween (PBST); the blocking buffer comprises bovine serum albumin solution, and the bovine serum albumin solution uses the PBS as a solvent; the chromogenic substrate comprises a 3,3′,5,5′-tetramethylbenzidine (TMB) solution; and the stop buffer comprises an aqueous sulfuric acid solution.
